(19) United States
(12) Patent Application Publication
(10) Pub. No.: US 2026/0158250 A1
(43) Pub. Date: Jun. 11, 2026

(54) THERAPEUTIC DISPENSING HEAD

(71) Applicant: SHENZHEN NUON MEDICAL EQUIPMENT CO., LTD., Shenzhen (CN)

(72) Inventors: ALAIN DIJKSTRA, Amstelveen (NL); LI XIANG, Shenzhen (CN); Tang Yuanzhou, Shenzhen (CN); Huang Jianlong, Shenzhen (CN); Li Yubao, Shenzhen (CN)

(73) Assignee: SHENZHEN NUON MEDICAL EQUIPMENT CO., LTD., Shenzhen (CN)

(21) Appl. No.: 19/406,838

(22) Filed: Dec. 2, 2025

(30) Foreign Application Priority Data
Dec. 6, 2024 (CN) 202411796580X
Dec. 6, 2024 (CN) 2024230155212
Oct. 22, 2025 (CN) 2025222362662

(51) Int. Cl. A61M 35/00; A45D 34/04; A61H 15/00; A61H 15/02; A61H 23/02; A61N 5/06

(52) U.S. Cl. CPC A61M 35/003; A45D 34/04; A61H 15/02; A61H 23/0245; A61N 5/0616; A45D 2200/155; A61H 2015/0014; A61H 2201/105; A61M 2205/051; A61M 2205/3368; A61M 2205/3606; A61M 2205/3633; A61M 2205/3653; A61M 2205/8206; A61N 2005/0651; A61N 2005/0663

(57) ABSTRACT

The invention provides a skincare dispensing device comprising a bottle body and a detachable dispensing head designed to heat only the skincare liquid present inside a flow channel. The dispensing head includes a multi-layer tube assembly with a first tube body, a second tube body, a sealing structure, and an elastic clamping member that secures a heating lead while preventing leakage. A heating element is arranged within or adjacent to the flow channel to rapidly warm the liquid, while a circuit board and temperature sensor regulate heating for safety and user comfort. The second tube body offers thermal insulation and conceals the heating element. Stimulation unit features, such as a massage head, phototherapy, microcurrent, piezoelectric, or thermal therapy unit, etc., which can be integrated to enhance treatment and absorption. By heating only the dispensed amount, the device reduces waiting time, preserves ingredient integrity, and improves overall skincare application efficiency.

### TECHNICAL FIELD

The present invention relates to the field of skincare product dispensing systems, particularly to heated liquid dispensing structures and temperature-controlled skincare containers, such as a therapeutic dispensing head. More specifically, it concerns dropper or pump, such as tube assemblies with integrated heating elements and stimulation applicators for delivering warmed skincare formulations to improve user comfort and application efficiency.

### BACKGROUND ART

Conventional skincare product containers and dispensing devices, such as droppers, pump bottles, squeeze tubes, and roller applicators, generally deliver cosmetic liquids at ambient temperature. While these devices allow basic dispensing, they cannot provide temperature control or stimulation features during application. Users often experience discomfort when applying cold skincare products, especially in winter or in air-conditioned environments, which can reduce product absorption efficiency and negatively affect user experience. Some devices attempt to incorporate heating elements into the bottle or cap; however, these designs typically heat the entire stored liquid. This leads to slow heating time, unnecessary energy consumption, repeated thermal cycling of the entire formulation, and potential degradation of heat-sensitive skincare ingredients.

Furthermore, existing heated applicators lack effective sealing structures, resulting in leakage risks, poor thermal insulation, and accidental exposure of heating wires. Devices with stimulation components, such as vibration heads or phototherapy elements, are usually standalone therapeutic devices and are not structurally integrated with a skincare dispensing mechanism. As a result, users must rely on separate tools for heating, massage, and liquid application, making the skincare routine inefficient and inconvenient.

Accordingly, there exists a gap in the market for a compact, efficient, and reliable skincare dispensing system capable of locally heating only the dispensed amount of skincare solution, while maintaining safety, preventing leakage, and optionally providing stimulation enhancements such as massage, hot-compress, microcurrent stimulation, or phototherapy etc. There is also a need for a temperature-controlled dispensing head that can be easily mounted onto conventional bottle bodies without requiring users to operate multiple devices.

The present invention addresses these shortcomings by providing a specialized dispensing head with an integrated multi-layer tube assembly and a precisely controlled heating structure, designed to heat only the skincare liquid contained within the flow channel rather than the entire bottle. This minimizes thermal load, reduces waiting time, and preserves the formulation's integrity. The invention incorporates advanced sealing structures, elastic clamping components, heating leads, routing grooves, temperature sensors, and stimulation units, all within a compact and detachable dispensing head. Through these improvements, the invention enhances heating efficiency, improves user comfort, promotes absorption of skincare formulations, and provides a multifunctional skincare experience using a single integrated device.

### OBJECTS OF THE INVENTION

Some of the objects of the invention are as follows:

An object of the present invention is to provide a skincare dispensing device with an integrated heating mechanism that warms only the liquid present within the flow channel, thereby delivering skincare formulations at a comfortable temperature without heating the entire bottle.

Another object of the invention is to provide a multi-layer tube assembly incorporating a first tube body, a second tube body, sealing components, and elastic clamping structures that ensure leak-proof operation, secure routing of heating leads, and improved thermal insulation.

A further object of the invention is to provide a dispensing head equipped with a compact electrical assembly, including a battery, circuit board, switch button, and optional charging terminal, enabling safe and efficient power management for the heating element.

Yet another object of the invention is to provide a heating element and temperature sensor configuration capable of rapidly raising the temperature of the skincare liquid, while enabling precise temperature control to prevent overheating and ensure safe user application.

An additional object of the invention is to provide a stimulation unit, such as a massage head or thermal therapy applicator, that may deliver supplementary treatments including hot-compress, vibration, microcurrent, or phototherapy to enhance product absorption and improve skincare outcomes.

Another object of the invention is to provide a dispensing head that is modular and detachable, allowing users to combine the heated dispensing head with standard bottle bodies for convenient replacement, cleaning, and compatibility with diverse skincare formulations.

A further object of the invention is to provide a dropper or press-pump-based dispensing structure that enables users to draw skincare product into the heating chamber, temporarily store it, warm it, and dispense it smoothly and efficiently.

Yet another object of the invention is to offer a compact, user-friendly, and aesthetically refined device whose enclosed heating architecture conceals the heating element, prevents accidental contact, reduces burn risk, and maintains the premium appearance of the skincare container.

Still another object of the invention is to provide a temperature-retaining and energy-efficient design, wherein only a small volume of the cosmetic liquid is heated on demand, thereby reducing heating time, minimizing energy consumption, and preventing degradation of heat-sensitive ingredients.

### SUMMARY OF THE INVENTION

According to a first aspect of the present invention, a therapeutic dispensing head for delivering a skincare liquid is provided. The therapeutic dispensing head comprises: a first tube body defining a flow channel configured to receive the skincare liquid; a second tube body disposed around at least a portion of the first tube body to form a multi-layer tube assembly; a heating element arranged within or adjacent to the flow channel and configured to heat only the skincare liquid present within the flow channel; a sealing ring positioned between the first and second tube bodies to reduce leakage; and an elastic clamping member positioned at a lower end of the multi-layer tube assembly, the elastic clamping member securing a heating lead and clamping the tube bodies together.

In one embodiment of the invention, the heating element comprises a resistive heating wire, a film heater, or a micro-heating chip.

In one embodiment of the invention, the heating element is positioned in contact with an outer surface of the first tube body to promote rapid thermal conduction.

In one embodiment of the invention, the sealing ring comprises an O-ring, gasket, or elastomeric sealing component.

In one embodiment of the invention, the elastic clamping member is formed of a flexible polymer or silicone material.

In one embodiment of the invention, the elastic clamping member further comprises a lead-receiving groove configured to hold the heating lead in position.

In one embodiment of the invention, the therapeutic dispensing head further comprises a stimulation unit arranged at an upper end of the tube assembly.

In one embodiment of the invention, the stimulation unit comprises a metallic, ceramic, or polymer applicator configured to contact a user's skin.

In one embodiment of the invention, the second tube body is composed of a thermally insulating material configured to retain heat within the first tube body.

According to a second aspect of the present invention, a therapeutic dispensing head configured to convey a skincare liquid is provided. The dispensing head comprises: a first tube body defining a first flow channel; a second tube body arranged around at least a portion of the first tube body and defining at least one secondary flow channel; a sealing structure positioned between the tube bodies to reduce leakage; a heating element arranged within or adjacent to the secondary flow channel and configured to heat the skincare liquid present within the first flow channel; and an elastic clamping member arranged at a lower end of the second tube body to hold the first tube body in position.

In one embodiment of the invention, the secondary flow channel accommodates the heating element, and the skincare liquid passes through a conduction channel of the heating element.

In one embodiment of the invention, the sealing structure comprises an annular sealing ring positioned between the first and second tube bodies.

In one embodiment of the invention, the elastic clamping member comprises a ring-shaped elastic structure configured to exert radial pressure on the first tube body.

In one embodiment of the invention, the first and second tube bodies together form a multi-layer tube assembly configured to guide the skincare liquid toward an outlet region.

According to a third aspect of the present invention, a therapeutic dispensing head for delivering a skincare liquid is provided. The dispensing head comprises: a flow channel for receiving the skincare liquid; a tube assembly including at least a first tube body forming the flow channel and a second tube body arranged around the first tube body; at least one sealing component disposed between the tube bodies; a heating element arranged within or adjacent to the flow channel and configured to heat only a portion of the skincare liquid; a temperature sensor and a circuit control module configured to regulate operation of the heating element; and a clamping or retention member configured to secure a heating lead.

In one embodiment of the invention, the temperature sensor comprises a thermistor or a temperature-sensitive integrated circuit.

In one embodiment of the invention, the circuit control module is configured to regulate the heating element based on a predetermined temperature range.

In one embodiment of the invention, the circuit control module includes a timing or power-limiting mechanism to prevent overheating.

In one embodiment of the invention, the clamping or retention member comprises an elastic ring, groove, or support bracket configured to hold the electrical lead.

In one embodiment of the invention, the therapeutic dispensing head further comprises a stimulation unit or applicator arranged at a dispensing end of the flow channel.

In the context of the specification, when an element is referred to as being “fixed to” or “disposed to” another element, it may either be directly on another element or indirectly on that other element. When a component is said to be “connected” or “connected to” another component, it may be directly connected to another component or indirectly connected to other components on the piece.

In the context of the specification, the terms “first”, “second,” and “third” are only used for descriptive purposes and do not imply the relative importance or implicitly indicate the quantity of technical features indicated.

In the context of the specification, the term “plurality” means two or more than two, unless otherwise indicated.

In the context of the specification, the term “several” means more than one, unless otherwise specified.

In the context of the specification, the term “handheld therapy device” refers to any device configured to emit therapeutic light for skin treatment, pain relief, or wellness applications.

In the context of the specification, the term “stimulation element” refers broadly to any component, module, or structure configured to apply a therapeutic or cosmetic stimulus to a user's skin or tissue. Stimulation elements may include, but are not limited to, phototherapy elements, massage elements, microcurrent electrodes, ultrasonic transducers, heating elements, cooling elements, or combinations thereof.

In the context of the specification, the term “phototherapy element” encompasses any light-emitting device capable of emitting light of therapeutic wavelength(s), including but not limited to light-emitting diodes (LEDs), organic LEDs (OLEDs), laser diodes, or equivalent optical sources. The light may include ultraviolet, visible, near-infrared, or far-infrared spectra.

In the context of the specification, the term “massage element” refers to any component adapted to apply mechanical stimulation to the skin, including rotating rollers, kneading members, vibrating members, or reciprocating structures. The massage element may be fixed, detachable, or mounted for rotation or vibration relative to the housing.

In the context of the specification, the term “microcurrent element” refers to any electrode or conductive structure configured to deliver a controlled electrical signal to the user's skin. Such elements may include paired electrodes, conductive surfaces, or pads connected to a circuit board for generating microcurrent, galvanic current, or equivalent electrical therapy.

In the context of the specification, the term “housing” is intended to cover any casing, enclosure, or structural body that contains or supports components of the device. The housing may include a handle portion, head portion, or other segments, and may be made from polymeric, metallic, composite, or other suitable materials.

In the context of the specification, the terms “head” or “phototherapy head” refer to a portion of the device coupled to the housing and configured to emit light toward the skin. The head may include one or more light-transmitting surfaces, optical lenses, or diffusers, and may also support electrodes or other stimulation elements.

In the context of the specification, the term “control interface” refers to any input or output mechanism enabling a user to operate the device. The control interface may include physical buttons, capacitive touch sensors, sliders, switches, or graphical displays, and may further include wireless control via a mobile application.

In the context of the specification, the term “circuit board” encompasses any printed circuit board (PCB), flexible circuit, or equivalent substrate that supports and electrically connects components of the device, including power supplies, control chips, drivers, or stimulation elements.

In the context of the specification, the term “user” or “subject” is intended to broadly cover humans, animals, or other recipients of the treatment, unless otherwise specifically limited.

In the context of the specification, the term “LED module” refers to one or more light-emitting diode (LED) elements that are electrically connected and configured to emit light of specific wavelengths suitable for therapeutic purposes. The LED module may include drive circuitry, heat dissipation structures, and optical elements such as lenses or diffusers to control light distribution.

In the context of the specification, the term “light source” or “phototherapy source” etc. refers to a source emitting coherent laser light, or light-emitting diodes (“LEDs”). The term “light therapy” refers to light generated from any of the sources, such as lasers, LED sources, or Super luminous diodes (“SLD”).

In the context of the specification, “Light Emitting Diodes (LEDs)” refer to semiconductor diodes capable of emitting electromagnetic radiation when supplied with an electric current. The LEDs are characterized by superior power efficiencies, smaller sizes, rapid switching speeds, physical robustness, and longer lifespans compared to incandescent or fluorescent lamps. The one or more LEDs may include through-hole type LEDs (generally emitting electromagnetic radiation in red, green, yellow, blue, and white colors), Surface Mount Technology (SMT) LEDs, Bi-color LEDs, Pulse Width Modulated RGB (Red-Green-Blue) LEDs, and high-power LEDs, among others.

Materials used in one or more LEDs may vary from one embodiment to another, depending upon the frequency of radiation required. Different frequencies can be obtained from LEDs made from pure or doped semiconductor materials. Commonly used semiconductor materials include nitrides of Silicon, Gallium, Aluminum, Boron, Zinc Selenide, etc., in pure form or doped with elements such as Aluminum and Indium. For example, red and amber colors are produced from Aluminum Indium Gallium Phosphide (AlGaInP) based compositions, while blue, green, and cyan use Indium Gallium Nitride based compositions. White light may be produced by mixing red, green, and blue lights in equal proportions, while varying proportions may be used to generate a wider color gamut. White and other colored lightings may also be produced using phosphor coatings such as Yttrium Aluminum Garnet (YAG) in combination with a blue LED to generate white light, and Magnesium-doped potassium fluorosilicate in combination with a blue LED to generate red light.

In addition to conventional mineral-based LEDs, one or more LEDs may also be provided on an Organic LED (OLED) based flexible panel or an inorganic LED-based flexible panel. Such OLED panels may be generated by depositing organic semiconducting materials over Thin Film Transistor (TFT) based substrates. Further, a discussion on the generation of OLED panels can be found in Bardsley, J. N (2004), “International OLED Technology Roadmap”, IEEE Journal of Selected Topics in Quantum Electronics, Vol. 10, No. 1, that is included herein in its entirety, by reference. An exemplary description of flexible inorganic light-emitting diode strips can be found in U.S. Pat. No. 7,476,557 B2, titled “Roll-to-roll fabricated light sheet and encapsulated semiconductor circuit devices”, which is included herein in its entirety by reference.

In the context of this specification, terms like “light”, “radiation”, “irradiation”, “emission” and “illumination”, etc. refer to electromagnetic radiation in frequency ranges varying from the Ultraviolet (UV) frequencies to Infrared (IR) frequencies and wavelengths, wherein the range is inclusive of visible light, UV and IR frequencies and wavelengths. It is to be noted here that UV radiation can be categorized in several ways depending on respective wavelength ranges, all of which are envisaged to be under the scope of this invention. For example, UV radiation can be categorized as Hydrogen Lyman-α (122-121 nm), Far UV (200-122 nm), Middle UV (300-200 nm), and Near UV (400-300 nm). The UV radiation may also be categorized as UVA (400-315 nm), UVB (315-280 nm), and UVC (280-100 nm). Similarly, IR radiation may also be categorized into several categories according to respective wavelength ranges, which are again envisaged to be within the scope of this invention. A commonly used subdivision scheme for IR radiation includes Near IR (0.75-1.4 μm), Short-Wavelength IR (1.4-3 μm), Mid-Wavelength IR (3-8 μm), Long-Wavelength IR (8-15 μm), and Far IR (15-1000 μm).

Unless otherwise stated, the term “light” as used in this specification encompasses electromagnetic radiation in the visible (380-780 nm) and infrared (780 nm-1000 nm) ranges, particularly red light (620-750 nm) and near-infrared (750-1400 nm) wavelengths commonly used in photobiomodulation therapy. Particular wavelengths which may be selected as the dominant emissive wavelength may include the follow, without any preference to be indicated by order: 400 nm, 405 nm, 420 nm, 430 nm, 450 nm, 465 nm, 515 nm, 530 nm, 532 nm, 590 nm, 630 nm, 633 nm, 640 nm, 650 nm, 655 nm, 660 nm, 670 nm, 680 nm, 780 nm, 785 nm, 810 nm, 830 nm, 840 nm, 850 nm, 860 nm, 870 nm, 904 nm, 915 nm, 980 nm, 1015 nm, 1060 nm, 1065 nm, 1070 nm, 1200, and 1400 nm. As used herein, the term “light therapy” refers to the use of one or more light sources of any type that emit light with a wavelength between about 400 and 1400 nm. The device may also emit blue or ultraviolet light for surface-level treatments such as acne reduction or microbial control.

The red light (approximately 630-660 nm) penetrates deeply into the scalp to stimulate blood circulation and enhance hair follicle activity, thus promoting hair growth and repair. Blue light (around 415-470 nm) exhibits antibacterial properties and is effective in treating scalp acne and reducing inflammation. Green light (approximately 520-540 nm) can help reduce pigmentation and soothe sensitive or irritated scalp tissue. Yellow light (around 580-600 nm) improves oxygen exchange in the cells and aids in detoxifying the scalp, while near-infrared light (800-850 nm) reaches deeper layers to accelerate healing and reduce pain.

### BRIEF DESCRIPTION OF THE ACCOMPANYING DRAWINGS

The accompanying drawings illustrate the best mode for carrying out the invention as presently contemplated and set forth hereinafter. The present invention may be more clearly understood from a consideration of the following detailed description of the preferred embodiments taken in conjunction with the accompanying drawings, wherein like reference letters and numerals indicate the corresponding parts in various figures in the accompanying drawings, and in which:
FIG. 1 illustrates a cosmetic bottle with a liquid dispensing head, in accordance with an embodiment of the present invention.

FIG. 2 illustrates a cross-sectional view of the cosmetic bottle with the liquid dispensing head, with a section C, in accordance with an embodiment of the present invention.

FIG. 3 illustrates an enlarged view of section C illustrated in FIG. 2, in accordance with an embodiment of the present invention.

FIG. 4 illustrates an exploded view of the cosmetic bottle and the liquid dispensing head, in accordance with an embodiment of the present invention.

FIG. 5 illustrates an exploded view of a pipe body assembly and a heating element, in accordance with an embodiment of the present invention.

FIG. 6 illustrates a top view of an elastic clamping member, in accordance with an embodiment of the present invention.

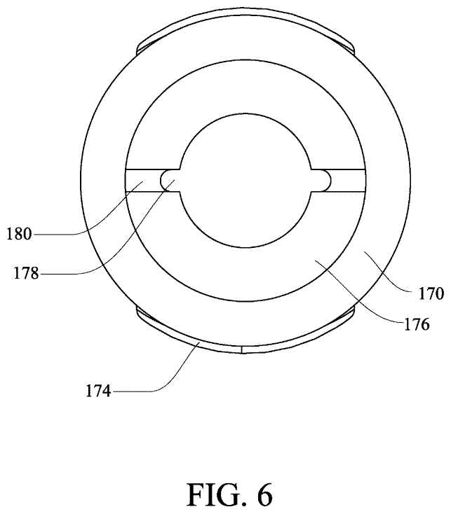

FIG. 7 illustrates a cross-sectional view of an alternate configuration of a cosmetic bottle and a liquid dispensing head, in accordance with an embodiment of the present invention.

FIG. 8 illustrates a cross-sectional view of an alternate configuration of a liquid dispensing head, in accordance with an embodiment of the present invention.

FIG. 9 illustrates a bottom view of the cosmetic bottle and the liquid dispensing head, in accordance with an embodiment of the present invention.

FIG. 10 illustrates a cross-sectional view of a first tube body along with a dropper cap, showing a section D, in accordance with an embodiment of the present invention.

FIG. 11 illustrates an enlarged view of section D illustrated in FIG. 10, in accordance with an embodiment of the present invention.

FIG. 12 illustrates a perspective view of the first tube body and the dropper cap, in accordance with an embodiment of the present invention.

FIG. 13 illustrates a cross-sectional view of a second tube body, in accordance with an embodiment of the present invention.

### DETAILED DESCRIPTION

Embodiments of the present invention disclosure will be described more fully hereinafter with reference to the accompanying drawings in which like numerals represent like elements throughout the figures, and in which example embodiments are shown.

The detailed description and the accompanying drawings illustrate the specific exemplary embodiments by which the disclosure may be practiced. These embodiments are described in detail to enable those skilled in the art to practice the invention illustrated in the disclosure. It is to be understood that other embodiments may be utilized, and other changes may be made, without departing from the spirit or scope of the present disclosure. The following detailed description is therefore not to be taken in a limiting sense, and the scope of the present invention disclosure is defined by the appended claims. Embodiments of the claims may, however, be embodied in many different forms and should not be construed as limited to the embodiments set forth herein.

The terms “a” and “an” herein do not denote a limitation of quantity, but rather denote the presence of at least one of the referenced items. The terms “having”, “comprising”, “including”, and variations thereof signify the presence of a component.

The present invention relates to an advanced skincare dispensing system designed to deliver cosmetic formulations at an optimally warmed temperature while ensuring efficient, safe, and hygienic application. Conventional skincare containers typically dispense products at ambient temperature, resulting in discomfort during cold use and reduced absorption. The invention overcomes these limitations by integrating a localized heating mechanism directly within the dispensing head, enabling rapid, controlled warming of only the liquid present in the flow channel.

The dispensing head features a multi-layer tube assembly that includes a first tube body, a second tube body, sealing structures, and an elastic clamping member. These structural components work together to guide the flow of skincare liquid, secure the heating lead, and prevent leakage. A heating element is strategically positioned within or adjacent to the flow channel to directly warm the solution, while a temperature sensor and circuit board provide precise thermal regulation for user safety and consistent performance.

Further, the invention accommodates stimulation features, such as a massage head, phototherapy unit, thermal therapy unit, microcurrent unit, or other treatment elements, allowing users to simultaneously warm and apply skincare products while enhancing overall therapeutic benefit. By heating only the dispensed amount of treatment liquid instead of the entire bottle, the device reduces waiting time, prevents degradation of heat-sensitive ingredients, and significantly improves user comfort and skincare efficiency.

Embodiments of the present invention will now be described with reference to the FIGS.

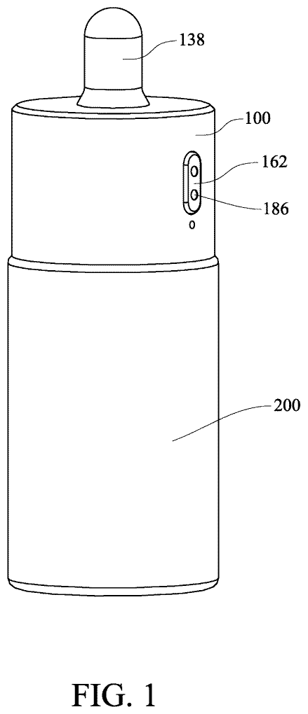

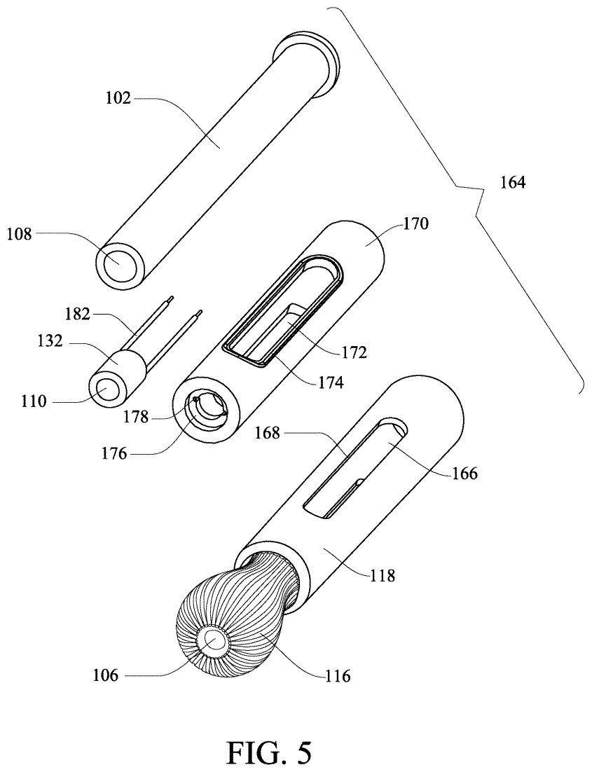

Referring to FIGS. 1 and 5, in one embodiment, the present invention provides a container comprising a bottle body 200 in combination with a dispensing head 100. The bottle body 200 is configured to store skincare formulations, while the dispensing head 100 is detachably mountable to the bottle body 200. This allows convenient replacement, cleaning, and modularity of use.

The dispensing head 100 further comprises a dropper cap 138 and a base assembly 184. A clearance hole 144 is formed on the base assembly 184, passing through the lid body 146, such that one end of the tube assembly 164 extends into the bottle body 200 while the opposite end passes through the clearance hole 144 and engages with the dropper cap 138.

Through this arrangement, the user may press and release the dropper cap 138 to induce suction, drawing the solution stored within the bottle body 200 into a flow channel 104. The drawn solution accumulates temporarily within the tube assembly 164 to form a temporary storage cavity, enabling the user to dispense the solution externally once the liquid discharge structure is removed from the bottle body 200.

The base assembly 184 includes a lid body 146 and an electrical assembly disposed therein. The clearance hole 144 is provided on the lid body 146, and the tube assembly 164 is fixedly or detachably connected to the lid body 146. The lower portion of the lid body 146 is detachably coupled to the bottle mouth of the bottle body 200. The detachable connection between the lid body 146 and the bottle mouth may be realized through a threaded engagement, a snap-fit interface, or an equivalent mechanical coupling.

In an embodiment, the electrical assembly comprises a battery 158, a circuit board 160, and a charging terminal 186. A switch button 162 is arranged on the lid body 146, and the battery 158, charging terminal 186, switch button 162, and heating lead 182 are all electrically connected to the circuit board 160. The charging terminal 186 is integrated with the switch button 162, thereby simplifying the external structure and improving overall compactness. The battery 158 supplies electrical energy to a heating element 132 through the circuit board 160, and the switch button 162 controls the activation and deactivation of the heating element 132.

In an embodiment, the shape and size of the dispensing head 100 can vary without affecting the functional performance of the other components/elements, such that the cover of the dispensing head 100 may be square, circular, rectangular, or of any other suitable shape.

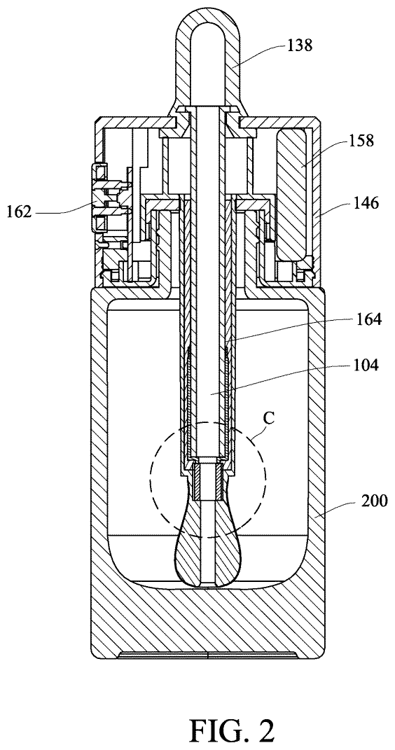

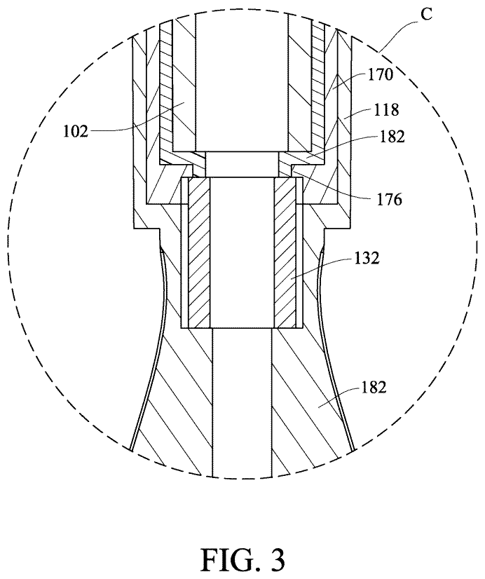

Referring particularly to FIGS. 2, 3, 5, and 7, in an embodiment, the dispensing head 100 comprises a tube assembly 164 and a heating element 132. The heating element 132 is configured to supply heat directly to the tube assembly 164, thereby heating the portion of the skin-care formulation contained therein. The tube assembly 164 includes a third segment 116, which can act as a stimulation unit, and the flow channel 104, the latter being formed within the tube assembly 164. A first end of the flow channel 104 extends into and communicates with the bottle body 200, thereby permitting the flow of the skin-care formulation into the flow channel 104. The flow channel 104 thus serves as a conduit for delivering and circulating the formulation. The stimulation unit is disposed at the first end of the tube assembly 164 and is configured to perform therapy on a user's skin.

The heating element 132 is positioned within the flow channel 104 such that it directly contacts the skin-care formulation flowing through the channel. By placing the heating element 132 internally within the flow channel 104, the present utility model limits the heated volume to only the liquid passing through the channel at a given moment. This structural arrangement minimizes thermal mass, reduces heat loss during transfer, and thereby enhances heating responsiveness and energy efficiency. As a result, the required heating time is substantially shortened compared to conventional containers that rely on heating the entire bottle.

The stimulation unit integrated on the third segment 116 is further configured to provide various forms of skin treatment. Depending on implementation, such stimulation functions may include, but are not limited to, phototherapy, microcurrent stimulation, vibration stimulation, thermal therapy (hot compress), cryotherapy (cold compress), roll-on massage, or combinations thereof. These stimulation functions may be selectively activated to improve the user's skincare experience while ensuring the dispensed product is delivered at an optimal temperature.

In an embodiment, the heating element 132 may be formed from an electrically insulating material exhibiting high thermal conductivity, such as ceramic, aluminium nitride, alumina, or similar materials. Such materials provide advantageous thermal transfer characteristics while maintaining electrical insulation. The heating element 132 may be configured as a tubular member such that the skin-care solution flows through an internal passage of the heating element 132 for direct and efficient heat exchange. In alternative embodiments, the heating element 132 may instead be formed as a solid rod, wherein an annular flow gap is defined between the outer surface of the rod and the inner wall of the flow channel 104, thereby enabling the solution to flow around the heating element 132 and absorb heat from its surface.

In an embodiment, the tube assembly 164 includes a first tube body 102 in which a first liquid-conduction channel 108 is formed. The first liquid-conduction channel 108 constitutes a portion of the overall flow channel 104. A second liquid-conduction channel 110 is further formed within the heating element 132. The liquid-outlet structure also comprises a first sealing ring 176 disposed within the flow channel 104. Along the axial direction of the flow channel 104, the first tube body 102, the first sealing ring 176, and the heating element 132 are arranged sequentially. One end of the first sealing ring 176 is sealingly coupled to the first tube body 102, while the opposite end of the first sealing ring 176 is sealingly coupled to the heating element 132, thereby ensuring fluid-tight communication between the first liquid-conduction channel 108 and the second liquid-conduction channel 110.

By providing the second liquid-conduction channel 110 within the heating element 132, the solution flows through a fully enclosed passage whose entire inner wall is in direct thermal contact with the heated structure. This configuration enables heat to be transferred radially inward from all sides of the solution, thereby minimizing localized heating and improving temperature uniformity across the flowing liquid. Additionally, the dual-end sealing configuration of the first sealing ring 176, which interfaces tightly with both the first tube body 102 and the heating element 132, effectively prevents leakage at the junction between these components. This arrangement maintains the integrity of the flow channel 104, reduces the likelihood of solution escape, and enhances the overall reliability of the liquid-dispensing structure.

In an embodiment, the dispensing head 100 may further include a temperature sensor, positioned within the flow channel 104 and electrically connected to the circuit board 160. The temperature sensor monitors the temperature of the solution within the flow channel 104 to ensure that the heating element 132 raises the solution temperature to a predetermined value. This configuration provides precise temperature regulation and enhances user comfort and safety during application.

The proposed dispensing head 100 is configured to draw a skin-care formulation stored in the bottle body 200 into the flow channel 104, where the formulation is subsequently subjected to localized heating. By heating, only the amount of liquid passing through the flow channel 104, rather than the entire volume contained in the bottle body 200, as in conventional devices, the present utility model significantly reduces heating time and improves overall heating efficiency.

In one embodiment, the tube assembly 164 further comprises an elastic clamping member 170. The elastic clamping member 170 is disposed around the outer periphery of the first tube body 102 and is securely connected to the outer wall thereof. The liquid-discharge structure also includes a heating lead 182, which is arranged externally along the first tube body 102. One end of the heating lead 182 is electrically connected to the heating element 132, while the opposite end is retained between the elastic clamping member 170 and the outer surface of the first tube body 102.

Through this arrangement, the elastic clamping member 170 serves to stably fix the heating lead 182 against the outer wall of the first tube body 102, thereby preventing displacement during use. Additionally, the elastic clamping member 170 provides a protective barrier that isolates the heating lead 182 from the cosmetic solution flowing within the first tube body 102, thus reducing the likelihood of fluid ingress and minimizing leakage-related risks.

In an embodiment, the heating element 132 comprises an insulating substrate in which a heating wire is embedded. The heating wire is electrically connected to the heating lead 182, enabling electrical power to be supplied thereto. Upon energization, the heating wire heats the surrounding insulating substrate, and the resulting thermal energy is transferred to the solution flowing through the flow channel 104.

In an embodiment, the heating element 132 may instead be formed as a conductive heating element. In such cases, the heating lead 182 is directly connected to the conductive heating element, and electrical power is supplied to heat the element. When the cosmetic solution in the flow channel 104 reaches a predetermined temperature, the conductive heating element may be de-energized, after which the heated solution is applied to the skin. The outer surface of the conductive heating element may be provided with an insulating layer to reduce electrical safety risks and further mitigate leakage.

Further, the elastic clamping member 170 is configured as a tubular structure and is connected to the first sealing ring 176. The tubular elastic clamping member 170 is sleeved over the exterior of the first tube body 102, and its inner diameter is smaller than the outer diameter of the first tube body 102.

The tubular configuration allows the elastic clamping member 170, in conjunction with the first sealing ring 176, to form a structure similar to a pen barrel that encircles the first tube body 102, thereby enhancing the sealing performance between the first sealing ring 176 and the first tube body 102. Because the inner diameter of the elastic clamping member 170 is smaller than the outer diameter of the first tube body 102, the elastic clamping member 170 must expand elastically when being fitted over the first tube body 102. Once installed, the inherent elastic recovery force of the elastic clamping member 170 causes it to contract and tightly grip the outer surface of the first tube body 102, thereby establishing a firm and reliable sealing interface. Consequently, the heating lead 182, which is positioned between the elastic clamping member 170 and the first tube body 102, is also securely sealed in place, effectively preventing exposure and reducing leakage pathways.

In an embodiment, the elastic clamping member 170 may be configured as a sheet-like structure. In such cases, the elastic clamping member 170 is connected to the first sealing ring 176 and is attached directly to the outer surface of the first tube body 102. By adopting a sheet configuration, the elastic clamping member 170 can precisely seal the localized region where the heating lead 182 is positioned, thereby reducing material consumption and enhancing assembly flexibility.

In an embodiment, a first mounting groove 178 is formed on the inner wall of the first sealing ring 176, and a second mounting groove 180 is formed on the end wall of the first sealing ring 176 adjacent to the first tube body 102. The second mounting groove 180 is in communication with the first mounting groove 178, and the heating lead 182 extends sequentially through the first mounting groove 178 and the second mounting groove 180. The first mounting groove 178 is oriented along the axial direction of the first sealing ring 176, whereas the second mounting groove 180 is oriented along the radial direction thereof. One end of the heating lead 182 is coupled to the heating element 132, while the opposite end, after passing through both the first mounting groove 178 and the second mounting groove 180, is retained between the elastic clamping member 170 and the first tube body 102.

The provision of the first mounting groove 178 and the second mounting groove 180 facilitates orderly routing of the heating lead 182, thereby reducing excessive compressive force on the first sealing ring 176 and the first tube body 102 during installation. This arrangement helps to minimize deformation of the first sealing ring 176, thereby preserving its sealing performance and improving structural reliability.

When the heating lead 182 passes through the first mounting groove 178 and the second mounting groove 180, it bends toward the interior of the elastic clamping member 170. Such a bend may otherwise present a potential leakage path. However, when the elastic clamping member 170 is configured as a tubular structure, it envelops the entire outer circumference of the first sealing ring 176, thereby enclosing the bent region of the heating lead 182 within its interior. This prevents exposure of the heating lead 182 around the outer surface of the first sealing ring 176, enhances protection of the heating lead 182, and effectively reduces the risk of leakage.

Referring further to FIG. 5, in an embodiment, the first sealing ring 176 is integrally molded with the elastic clamping member 170. Through such an integrally molded configuration, any interface gap between the first sealing ring 176 and the elastic clamping member 170 is eliminated, significantly reducing the likelihood of fluid leakage and improving overall sealing performance. Additionally, the integrated manufacturing structure simplifies assembly, reduces the number of installation steps, and improves assembly efficiency.

Referring to FIGS. 2 to 5, in an embodiment, the dispensing head 100 further comprises a second tube body 118. The second tube body 118 is sleeved around the exterior of the elastic clamping member 170, and the elastic clamping member 170 is sealingly connected to the inner wall of the second tube body 118. A fourth liquid-conduction channel 166 is formed within the second tube body 118, and is in fluid communication with the first liquid-conduction channel 108, collectively forming part of the flow channel 104. The heating element 132 is mounted within the fourth liquid-conduction channel 166.

With this configuration, the tube assembly 164 adopts a multi-layer tubular structure, which enhances the overall rigidity of the assembly and reduces deformation during use. The structural reinforcement helps maintain stable liquid flow within the flow channel 104. Moreover, the multi-layer arrangement provides improved thermal insulation around the solution flowing through the flow channel 104, thereby reducing heat loss and enhancing temperature retention. The positioning of the heating element 132 within the fourth liquid-conduction channel 166 ensures that the heating element 132 is enclosed and not exposed externally, which prevents accidental user contact and reduces the risk of burns.

In an embodiment, the outer wall of the heating element 132 may be in direct contact with the inner wall of the fourth liquid-conduction channel 166, such that the second liquid-conduction channel 110 formed within the heating element 132 also constitutes a portion of the liquid flow pathway. Alternatively, a clearance may be provided between the outer wall of the heating element 132 and the inner wall of the fourth liquid-conduction channel 166, allowing the solution to flow through the annular gap. In this arrangement, heat from both the inner and outer surfaces of the heating element 132 is transferred to the solution, thereby increasing the effective heat exchange area and improving the overall heating efficiency of the solution within the flow channel 104.

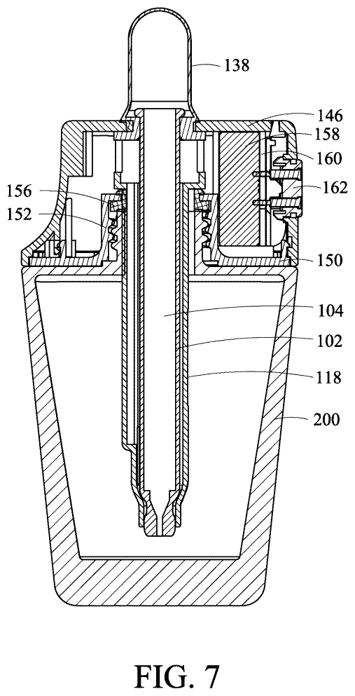

Referring to FIGS. 5 and 7, in an embodiment, the dispensing head 100 further comprises the second tube body 118. A perspective region is formed on the first tube body 102, a first avoidance hole 172 is provided on the elastic clamping member 170, and a second avoidance hole 168 is formed on the second tube body 118. The perspective region, the first avoidance hole 172, and the second avoidance hole 168 are arranged in alignment in the radial direction. A convex ring 174 is provided on the outer wall of the elastic clamping member 170, surrounding the periphery of the first avoidance hole 172. The convex ring 174 is sealingly connected to the inner wall of the second tube body 118.

The particular region may be a partially transparent or translucent section of the first tube body 102, or the entire first tube body 102 may be made of a transparent or translucent material. The specific degree of transparency is not limited.

Through this arrangement, the user may visually observe the liquid level within the flow channel 104 by viewing through the second avoidance hole 168, the first avoidance hole 172, and the particular region successively. This enables the user to judge the amount of skincare solution being delivered. When the second tube body 118 is assembled over the elastic clamping member 170, the convex ring 174 forms a sealed fit against the inner wall of the second tube body 118, thereby preventing the skincare solution from the bottle body 200 from entering the interior of the tube assembly 164 via the second avoidance hole 168. This reduces the risk of the heating lead 182 being exposed to liquid and minimizes short-circuit hazards.

Referring to FIGS. 2 to 5, in one embodiment, the third segment 116 is provided with a liquid inlet 106, which communicates with the flow channel 104. This allows the cosmetic solution within the flow channel 104 to be discharged through the liquid inlet 106, thereby enhancing the synergy between fluid delivery and the stimulation function. The third segment 116 facilitates solution application, aids in spreading the solution, and promotes absorption during user operation.

In an embodiment, the third segment 116 can be configured as detachable and multiple interchangeable therapeutic attachments. The third segment 116 can be removably coupled to the first tube body 102 or to the second tube body 118 through a threaded interface, bayonet lock, snap-fit connector, magnetic coupling, or equivalent mechanical structure, allowing users to select from multiple treatment heads according to their needs. The modular configuration of the third segment 116 enables the dispensing head 100 to adapt to a wide range of skincare and therapeutic requirements while maintaining compactness and ease of use. These additional interchangeable attachments enable users to customize their skincare routine and transform the dispensing head 100 into a multifunctional treatment tool.

In an embodiment, the stimulation unit of the third segment 116 is formed from a thermally conductive material and is positioned adjacent to the heating element 132. By locating the stimulation unit on the third segment 116 in close proximity to the heating element 132, heat generated by the heating element 132 can be effectively transferred to the stimulation unit, warming it to a comfortable temperature. This reduces skin irritation during contact, enhances the user's stimulation experience, and enables the stimulation unit to provide a localized hot-compress effect.

The third segment 116 and the second tube body 118 may be provided as separate components or as an integral molded structure. Preferably, the third segment 116 and the second tube body 118 are integrally formed, and at least part of the outer wall of the heating element 132 is positioned adjacent to the inner wall of the liquid inlet 106, ensuring direct and efficient heat transfer from the heating element 132 to the stimulation unit.

In an embodiment, the stimulation unit of the third segment 116 may be configured as a massage head, which may adopt various shapes such as drop-shaped, spherical, or bullet-shaped forms, without limitation. The outer surface of the massage head may be provided with guiding ridges or patterned lines to direct massage movement, reduce undesired friction, and protect skin tissues during use.

In an embodiment, the stimulation unit of the third segment 116 may incorporate one or more stimulation elements, which can include a phototherapy element, microcurrent element, heating element, cooling element, piezoelectric element, etc., or combinations thereof, without limitation. These stimulation elements can be integrated within or disposed of on the third segment to provide multiple therapeutic effects during user operation.

In an embodiment, the third segment 116 can be a stimulation element can be a transparent or translucent attachment which accommodates a phototherapy element configured to emit therapeutic light toward the user's skin. The phototherapy element is configured to emit light of specific wavelengths, such as red, blue, or near-infrared light, to promote skin rejuvenation, enhance microcirculation, or assist in the absorption of cosmetic solutions. The light emitted from the phototherapy element can penetrate the skin to stimulate cellular activity, support collagen production, and improve overall skin texture and tone.

In an embodiment, the third segment 116 can be a stimulation element, which can be an attachment to provide microcurrent therapy. The microcurrent element equipped with conductive electrodes can be installed to deliver low-intensity electrical currents that mimic the body's natural bioelectric signals. Application of microcurrents via the stimulation unit can stimulate facial or scalp muscles, enhance cellular metabolism, and promote absorption of topical solutions. Microcurrent therapy also improves skin firmness and elasticity over time.

In an embodiment, the third segment 116 can be a stimulation element, which can be an attachment to provide thermal therapy. A thermal therapy can be provided, incorporating a separate thermal element inside the attachment, or the adjacent heating element 132 can be used to deliver localized warm-compress treatment in conjunction with the warmed skincare formulation. The thermal element provides localized warmth to the stimulation unit, which can improve blood circulation, relax tissues, and reduce muscle tension. Heat generated by the thermal element can enhance penetration and absorption of cosmetic solutions, providing a hot-compress effect for user comfort.

The thermal element can also deliver a cold-compress effect, which can soothe irritated skin, reduce inflammation, and tighten pores. Cooling therapy can be particularly beneficial after phototherapy, microcurrent treatment, or application of active cosmetic formulations.

In an embodiment, the third segment 116 can be a stimulation element, which can be an attachment with a piezoelectric element. In an embodiment, a piezoelectric element may be provided to generate vibrational massage for enhanced absorption and localized stimulation. The piezoelectric element converts electrical signals into mechanical vibrations, allowing the stimulation unit to generate precise vibrational massage. These vibrations help in spreading cosmetic solutions evenly across the skin, improve local microcirculation, and provide gentle stimulation to the underlying tissues. Piezoelectric stimulation can be combined with other elements, such as heating or phototherapy, to achieve a synergistic therapeutic effect.

In an embodiment, the third segment 116 can be a massage roller attachment, which can include one or more rotating or fixed roller bodies configured to distribute the skincare product evenly while applying mechanical stimulation to enhance microcirculation and improve absorption.

In an embodiment, the third segment 116 is configured not only to facilitate treatment of the skincare liquid inside the first tube body 102 but also to deliver stimulation therapy when brought into contact with the user's skin. As the warmed liquid passes through or around the third segment 116, any integrated stimulation elements, such as the phototherapy element, the microcurrent element, the piezoelectric element, or the thermal element, etc., can be activated simultaneously by touching the user's skin. Moreover, when the user places the third segment 116 against the skin, the stimulation unit is activated either through a contact sensor, capacitive detection, or manual user input, thereby providing direct therapy to the targeted area. This dual-function design allows the third segment 116 to condition the liquid and to deliver localized stimulation during application, ensuring improved absorption, enhanced treatment efficacy, and a more comprehensive skincare experience.

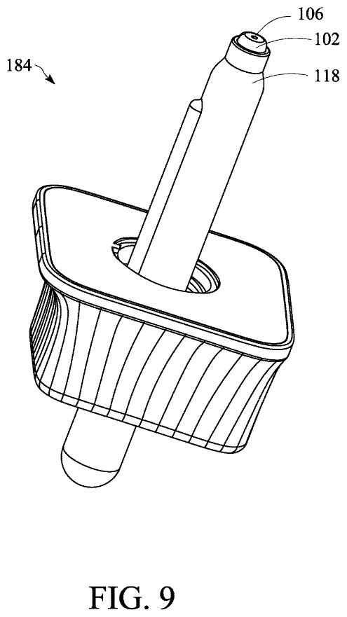

Referring to FIGS. 7 and 9, in an embodiment, an alternate configuration of the dispensing head 100 and the bottle body 200 is provided. The dispensing head 100 comprises a first tube body 102, a second tube body 118, and a heating element 132. The first tube body 102 defines a flow channel 104 for guiding the skincare product. The second tube body 118 is sleeved over the first tube body 102, creating a storage gap between them. The second tube body 118 is formed of a plastic material. The heating element 132 is disposed within the storage gap and is arranged in close contact with the outer circumferential surface of the first tube body 102.

In operation, the heating element 132 generates heat, which is transferred to the first tube body 102. As the skincare product flows through the flow channel 104, heat is conducted from the first tube body 102 to the skincare product, elevating its temperature before dispensing. This configuration ensures that the product is delivered at a comfortable temperature, reducing irritation caused by low-temperature application. The second tube body 118 functions as an insulating enclosure, retaining heat generated by the heating element 132, thereby reducing thermal loss, accelerating product heating, and improving user convenience. The plastic material of the second tube body 118, having relatively low thermal conductivity, further enhances heat retention. Additionally, the second tube body 118 conceals the heating element 132, improving overall aesthetics and preventing accidental impact damage. Because the heating element 132 heats only the product within the first tube body 102, the skincare product stored in the bottle body 200 remains unheated, preventing repeated heating cycles and reducing the risk of degradation.

The first tube body 102 and the second tube body 118 adopt a coaxial sleeve-type configuration. Their lengths may be identical or different. For example, the second tube body 118 may be shorter than the first tube body 102, extending only across the region corresponding to the heating element 132 and optionally over portions of the first tube body 102 positioned on both axial ends of the heating element 132.

The first tube body 102 may be formed from plastic, glass, or another material exhibiting a higher thermal conductivity than that of the second tube body 118. This enables efficient transfer of heat from the heating element 132 to the flow channel 104.

In an embodiment, the first tube body 102 and the second tube body 118 are formed of transparent materials, allowing visual observation of the skincare product dosage. In other embodiments, the outer surface of the second tube body 118 is coated or plated with a non-transparent layer while a viewing window 130 is reserved, enabling selective visibility of the internal product.

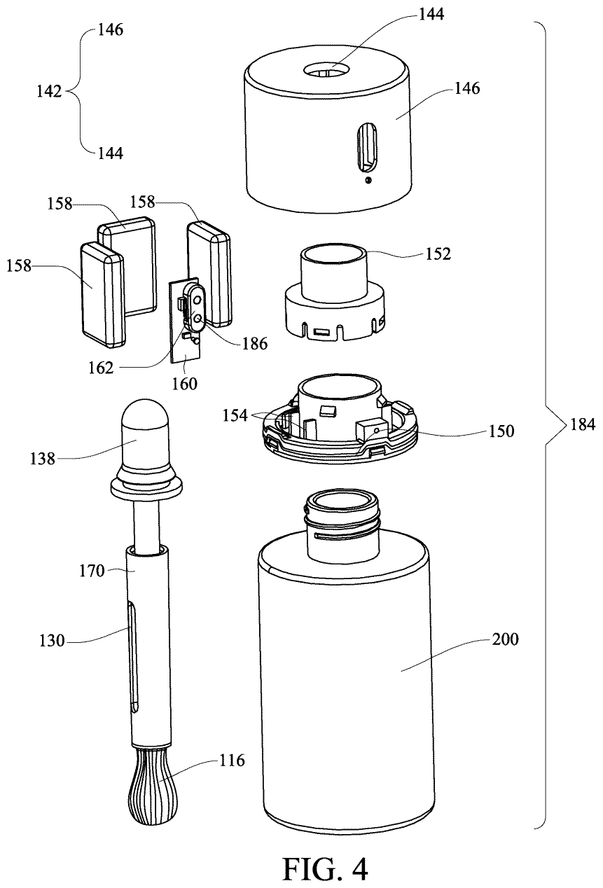

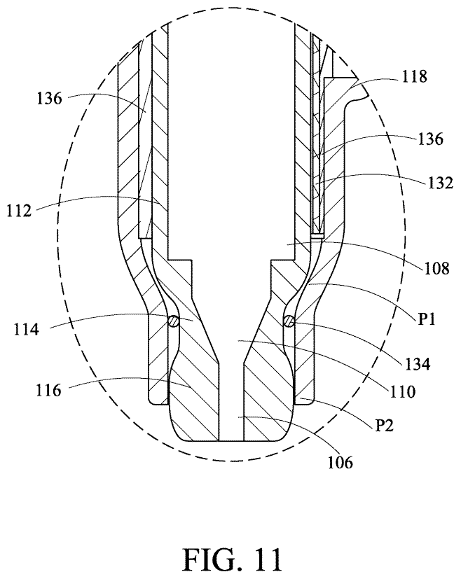

Referring to FIGS. 4, 9, 10, and 11, in an embodiment, the flow channel 104 includes a liquid inlet 106, and the heating element 132 is disposed adjacent to the liquid inlet 106. This arrangement concentrates heating at the region where the product enters the first tube body 102, thereby reducing thermal influence on electronic components housed in a cover 142. Additionally, when the dispensing head 100 operates in a dropper-type configuration, even a small quantity of product suctioned into the first tube body 102 accumulates near the liquid inlet 106, ensuring adequate heating.

The heating element 132 is positioned near the liquid inlet 106, in the region located between the axial center point of the heating element 132 and the liquid inlet 106, relative to the axial center line of the first tube body 102.

In an embodiment, the aperture of the liquid inlet 106 is smaller than the aperture of the flow channel 104, forming a constriction. In some implementations, the aperture of the liquid inlet 106 is substantially smaller, e.g., less than one-third of the diameter of the flow channel 104. The reduced aperture effectively suppresses heat escape from the flow channel 104, allowing heat to be concentrated within the channel for rapid product heating while limiting heat transfer back into the bottle body 200, thereby avoiding repeated heating of stored product.

In an embodiment, the aperture diameter of the liquid inlet 106 is in a range of 1.2-2 mm, such as 1.2 mm, 1.5 mm, 1.8 mm, or 2 mm. This range ensures smooth inflow and outflow of the skincare product while maintaining a sufficiently small opening to minimize heat loss.

In an embodiment, the second tube body 118 is at the axial end proximate the liquid inlet 106. This prevents the product from entering the storage gap between the tubes, thereby protecting the heating element 132 from corrosion and leakage. The sealed connection also inhibits heat from dissipating toward the liquid inlet 106, enabling the heat generated by the heating element 132 to accumulate within the storage gap and provide enhanced thermal insulation around the first tube body 102.

The seal between the first tube body 102 and the second tube body 118 may be achieved using a second sealing ring 134 or through the application of an adhesive sealing compound.

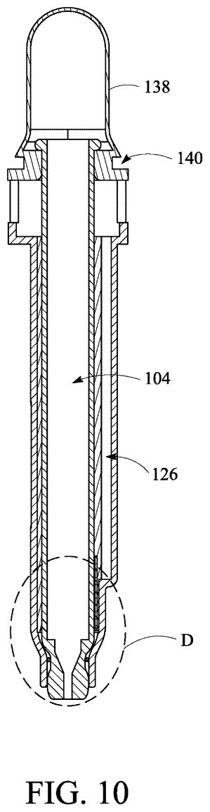

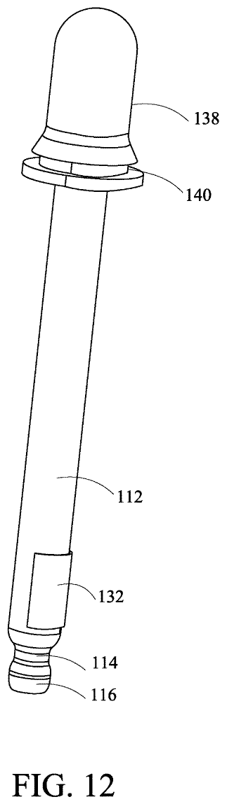

Referring to FIGS. 10 to 12, in an embodiment, in an alternate configuration of dispensing head 100, the first tube body 102 comprises a first segment 112, a second segment 114, and a third segment 116, sequentially arranged along the axial direction. The outer diameter of the first segment 112 is greater than that of the third segment 116. The liquid inlet 106 is formed in the third segment 116, ensuring that the wall thickness at the inlet region remains relatively small, which facilitates manufacturing and reduces material use.

In an embodiment, the outer diameter of the second segment 114 gradually decreases and then gradually increases along the axial direction from the first segment 112 toward the third segment 116. The dispensing head 100 further comprises the second sealing ring 134, which is sleeved over the second segment 114 and forms a sealing engagement between the second segment 114 and the second tube body 118. In this embodiment, the outer circumferential surface of the second segment 114 includes an annular groove configured to accommodate the second sealing ring 134, thereby preventing axial displacement of the second sealing ring 134. The gradual variation of the outer diameter of the second segment 114 avoids abrupt geometrical transitions in the first tube body 102, facilitating molding of the first tube body 102 and preventing stress concentrations that may otherwise result from sudden dimensional changes.

The second tube body 118 is tightly fitted onto the first tube body 102 and includes a first abutting position P1 and a second abutting position P2, each abutting the first tube body 102. The first abutting position P1 is located at the junction between the first segment 112 and the second segment 114, while the second abutting position P2 is located at the junction between the second segment 114 and the third segment 116. In this embodiment, the dimensions of the second tube body 118 correspond substantially with the contour of the first tube body 102, thereby minimizing excessive clearance between the tubes. The first abutting position P1, and the second abutting position P2 are arranged on opposite sides of the second sealing ring 134, thus preventing the second sealing ring 134 from leaving the second segment 114 and ensuring a reliable seal between the first tube body 102 and the second tube body 118. Additionally, the second abutting position P2 forms a first sealing interface adjacent to the liquid inlet 106, preventing the skincare product from entering the gap between the second segment 114 and the second tube body 118. The connection between the first segment 112 and the second segment 114 further forms a step structure, limiting axial movement of the second tube body 118 relative to the first tube body 102.

In an embodiment, the flow channel 104 comprises a first liquid-conduction channel 108, located within the first segment 112, and a second liquid-conduction channel 110, located within the second segment 114. The aperture of the first liquid-conduction channel 108 is larger than the aperture of the second liquid-conduction channel 110, and the second liquid-conduction channel 110 gradually narrows along the axial direction from the first segment 112 toward the third segment 116. When the skincare product enters through the liquid inlet 106, it is discharged into the second liquid-conduction channel 110 and subsequently accumulates within the larger first liquid-conduction channel 108, thereby preventing clogging at the liquid inlet 106. Moreover, the gradual change in the aperture of the flow channel 104 is coordinated with the outer diameter profile of the first tube body 102.

The heating element 132 is mounted on the first segment 112, with one axial end of the heating element 132 positioned adjacent to the junction between the first segment 112 and the second segment 114, corresponding to the first abutting position P1. Accordingly, the first abutting position P1 not only reduces heat dissipation from the heating element 132 toward the second abutting position P2, but also provides a positional restraint for the heating element 132, preventing it from shifting toward the direction of P2.

The heating element 132 has a length significantly shorter than the length of the first segment 112. This ensures that the heating element 132 is spaced apart from the cover 142, preventing heat transfer to the electronic components located within the cover 142 and reducing the risk of overheating of such components. In some embodiments, the length of the heating element 132 is less than half of the axial length of the first segment 112.

The heating element 132 is in thermal contact with the first tube body 102. The heating element 132 has a curvature substantially matching that of the outer circumferential surface of the first tube body 102, thereby providing an enlarged heat-transfer contact area. The heating element 132 may directly contact the first tube body 102, or a thermally conductive grease may be interposed therebetween to enhance heat conduction.

Furthermore, a heat insulation layer 136 is disposed within the storage gap and surrounds both the heating element 132 and the first tube body 102. The heat insulation layer 136 is formed of a thermally insulating material, such as heat-insulating cotton or a plastic material having a lower thermal conductivity than the second tube body 118. The heat insulation layer 136 thermally isolates the heating element 132, significantly reducing heat loss and enabling rapid temperature rise within the first tube body 102, thereby reducing the user's waiting time.

In an embodiment, the heat insulation layer 136 has a cylindrical configuration extending across both axial ends of the heating element 132 and is sleeved over the first tube body 102. The heat insulation layer 136 maintains constant contact between the heating element 132 and the outer circumference of the first tube body 102, thereby facilitating efficient heat transfer. Due to the positional constraint provided by the heat insulation layer 136, no additional fastening structure is required to secure the heating element 132 to the first tube body 102.

In an embodiment, the heating element 132 is implemented as a heating plate. The heating plate may be configured as a closed annular structure or, in alternative examples, may adopt a non-closed arc-shaped profile, such as a highly curved arcuate heating plate. In an embodiment, the heating element 132 may be formed as a heating wire, for example, a spiral-shaped heating wire.

For enhanced thermal coupling, in an embodiment, the heating element 132 is disposed circumferentially around the first tube body 102. When multiple heating elements 132 are provided, they are arranged in a distributed manner around the periphery of the first tube body 102, thereby ensuring uniform heating.

The heating element 132 is retained between the first tube body 102 and the heat insulation layer 136 by virtue of the tight fit formed among the first tube body 102, the second tube body 118, and the heat insulation layer 136. Consequently, no separate fixing component or structural member is required to secure the heating element 132 to the first tube body 102.

Furthermore, the inner wall of the second tube body 118 is provided with a stepped surface. A portion of the heating element 132, specifically the end adjacent to the liquid inlet 106, is positioned so as to overlap this stepped surface. The stepped surface provides axial positioning for the heating element 132 within the first tube body 102, supporting it and preventing axial displacement toward the liquid inlet 106. Simultaneously, the heat insulation layer 136, which wraps around both the heating element 132 and the first tube body 102, further restricts movement of the heating element 132, ensuring stable placement during use.

As an alternative configuration, in an embodiment, the second tube body 118 is formed with a bent region near the end adjacent to the liquid inlet 106. This bent structure accommodates variations in the outer diameter of the first tube body 102, enabling the receiving gap to be narrowed in the vicinity of the first contact point P1. By reducing the width of the receiving gap near this region, accidental detachment or downward displacement of the heating element 132 is prevented.

Since the heating element 132 generates heat upon energization, localized heat accumulation may occur. To prevent overheating and potential safety hazards, a temperature sensor may be provided between the first tube body 102 and the second tube body 118 in some embodiments. When an excessive temperature is detected, the circuit board 160 automatically interrupts the power supply to the heating element 132. Conversely, when the sensed temperature falls below a predetermined threshold, the circuit board 160 increases the current supplied to the heating element 132, thereby raising the temperature of the skincare product.

In practical applications, heating the skincare product to approximately 40° C., for example, within a range of 38-45° C., effectively mitigates cold discomfort during application. Considering thermal losses and environmental influences, an upper limit of 50° C. and a lower limit of 38° C. may be defined as safety thresholds. When the temperature sensor detects a temperature exceeding 50° C., the circuit board 160 shuts off the heating element 132. When the detected temperature falls below 38° C., the circuit board 160 increases the current supply to elevate the heating temperature.

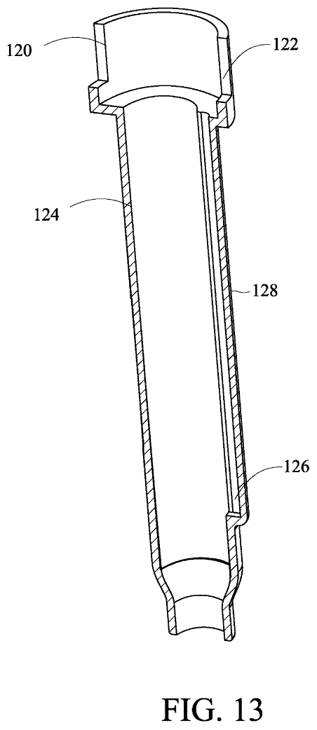

Referring to FIG. 13, the inner wall of the second tube body 118 is provided with a mounting groove 126, which extends to one end of the second tube body 118, specifically the end opposite the liquid inlet 106. A temperature sensor is mounted within the mounting groove 126 such that it can be installed by insertion from the end of the second tube body 118 remote from the liquid inlet 106. In certain embodiments, a portion of the second tube body 118 is outwardly expanded to form a bulged region 128, thereby defining the mounting groove 126 on its inner wall.

In an embodiment, the dispensing head 100 adopts a dropper-type structure and is provided with a single liquid inlet 106, through which the skincare product is delivered from the bottle body 200 to the user.

Referring to FIG. 7, in an embodiment, in an alternate embodiment, the dispensing head 100 further includes a dropper cap 138. The dropper cap 138 is fixed to the ends of both the first tube body 102 and the second tube body 118. Accordingly, the end of the first tube body 102 furthest from the dropper cap 138 defines the liquid inlet 106. By squeezing the dropper cap 138, the skincare product is drawn into the flow channel 104 through the liquid inlet 106, where it may be temporarily retained until heated to a desired temperature. Subsequent squeezing of the dropper cap 138 causes the heated skincare product to be dispensed outwardly through the same liquid inlet 106.

In an embodiment, the dispensing head 100 further comprises a cover 142 provided with a clearance hole 144. The first tube body 102 and the second tube body 118 extend through the clearance hole 144 and protrude from one side of the cover 142. A dropper cap 138 is fixed to the ends of the first tube body 102 and the second tube body 118, positioned on the opposite side of the cover 142.

The cover 142 accommodates the battery 158 and a circuit board 160, and a switch button 162 is arranged on its exterior surface. The battery 158, circuit board 160, and heating element 132 are electrically connected. When the switch button 162 is actuated, the battery 158 supplies power to the heating element 132.

In an embodiment, the cover 142 comprises a lid body 146 and a bottom cover 150 coupled together to define an internal receiving space for housing the battery 158 and the circuit board 160. The clearance hole 144 is formed by a first hole on the lid body 146 and a second hole on the bottom cover 150. The dropper cap 138 is located adjacent to the lid body 146 and is provided with an annular groove 140. The edge of the first hole engages with the annular groove 140, thereby securing the dropper cap 138 to the lid body 146.

The bottom cover 150 is further provided with a heat-insulating wall 152, which surrounds the second hole and extends into the receiving space. The heat-insulating wall 152 isolates the battery 158, circuit board 160, and other electronic components from the second tube body 118, thereby preventing heat generated by the heating element 132 from being transferred to the electronic components.

An internal thread is formed on the inner circumferential surface of the heat-insulating wall 152, while the bottle mouth of the bottle body 200 is provided with a corresponding external thread. Engagement between the internal and external threads allows the bottle body 200 to be detachably secured to the cover 142. In an embodiment, the cover 142 may be connected to the bottle body 200 through a snap-fit mechanism.

A third sealing ring 156 is disposed at the end of the heat-insulating wall 152. The third sealing ring 156 includes an annular groove that engages the end of the heat-insulating wall 152.

Referring to FIG. 13, the second tube body 118 includes a large tube section 120 with a large diameter and a small tube section 124 with a small diameter, connected to form a stepped surface. This stepped surface abuts against the third sealing ring 156, while the third sealing ring 156 forms a sealed fit with the small tube section 124. Accordingly, the third sealing ring 156 prevents liquid stored within the bottle body 200 from leaking out through the second hole.

The large tube section 120 further includes a wire passage 122 configured to accommodate electrical wires extending between the circuit board 160 and the heating element 132, thereby enabling unobstructed electrical connectivity.

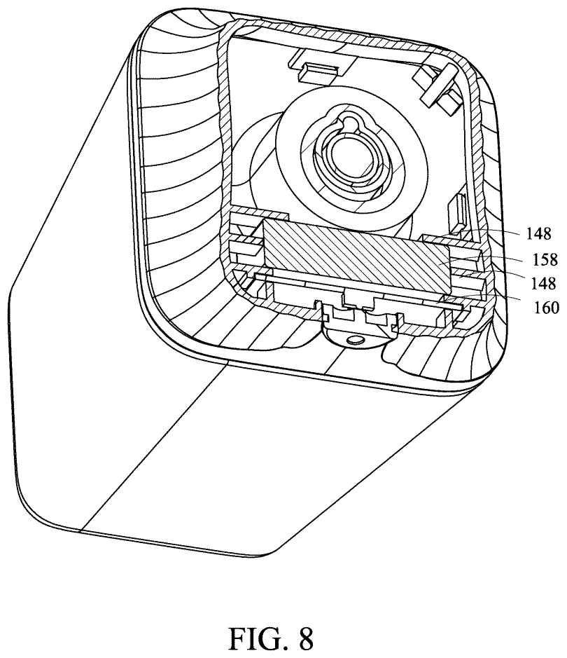

Referring to FIGS. 4, 7, and 8, the bottom cover 150 is provided with supporting ribs 154. The battery 158 and the circuit board 160 rest upon the supporting ribs 154, thereby forming a gap between these components and the side of the bottom cover 150 facing the lid body 146. This gap facilitates the circulation of heat generated by the battery 158 and the circuit board 160, thereby improving heat dissipation.

Furthermore, the lid body 146 can be provided with a plurality of limiting ribs 148, which form a groove-like accommodation space. The battery 158 and the circuit board 160 are received within this space, and the limiting ribs 148 contact their outer surfaces to restrict movement and maintain stable positioning.

In an embodiment, when the first tube body 102 and the second tube body 118 adopt a dropper configuration, the dispensing head 100 further includes a pump head. The pump head is positioned at the end of the first tube body 102, opposite the liquid inlet 106, and is movably connected to the cover 142. A single press of the pump head draws the skincare product from the bottle body 200 into the flow channel 104, where heating occurs. Subsequent pressing expels the skincare product through the liquid inlet 106.

In an embodiment, in another configuration, the dispensing head 100 may employ a press-pump mechanism and include both the liquid inlet 106 and the outlet. The first tube body 102 is accordingly formed with the liquid inlet 106 and a discharge outlet. A press-pump (not shown) is mounted on the cover 142. When the press-pump is actuated, the skincare product enters the flow channel 104 via the liquid inlet 106 and exits through the outlet. Because the product enters from one end of the first tube body 102 and exits from the other, the residence time within the flow channel is short, enabling rapid product dispensing.

This embodiment further provides a skincare product container comprising the bottle body 200 and the dispensing head 100, the structure of which is detailed in the foregoing description.

In an embodiment, the dispensing head 100 includes the cover 142 and the dropper cap 138, where the cover 142 defines the clearance hole 144 through which the first tube body 102 and the second tube body 118 extend and protrude toward the bottle body 200. The dropper cap 138 is affixed to the ends of the first tube body 102 and the second tube body 118, on the side of the cover 142 opposite the bottle body 200. The liquid inlet 106 located at the end of the first tube body 102 extends into the bottle body 200, such that squeezing the dropper cap 138 draws the skincare product into the flow channel 104.

The bottle body 200 may adopt various cross-sectional shapes, such as circular, elliptical, or polygonal. The bottle body 200 may also be formed with a contoured profile, for example, narrower at the top and bottom and wider in the middle. The particular configuration of the bottle body 200 is not limited to the present invention.

In an embodiment, the dispensing head 100 may further be equipped with a detection unit configured to determine whether the flow channel 104 contains skincare liquid. The detection unit may include, for example, an optical sensor, a capacitive sensor, a conductive sensor, or a pressure-based sensing element arranged along the first tube body 102 or within the flow channel 104. When the detection unit identifies that the tube is empty or that no liquid is present within the heating region, it transmits a signal to the circuit board 160. In response, the circuit board 160 prevents activation of the heating element 132 or immediately terminates its operation, thereby avoiding unnecessary heating and preventing potential overheating of the empty tube structure. The circuit board 160 allows operation of the heating element 132 only when the detection unit detects liquid within the first tube body 102. This configuration ensures safe, efficient, and intelligent temperature control while protecting both the device components and the skincare formulation.

In an embodiment, the dropper cap 138 and the detection unit can be configured to operate cooperatively to optimize heating efficiency and safety. When the user actuates the press the dropper cap 138 to draw skincare liquid from the bottle body 200 into the first tube body 102, the detection unit senses the presence of incoming liquid and transmits a corresponding signal to the circuit board 160. In response, the circuit board 160 enables activation of the heating element 132 so that the drawn liquid may be rapidly warmed within the flow channel 104. Conversely, when the dropper cap 138 is actuated again to dispense the warmed liquid outwardly, the detection unit detects the reduction or absence of liquid within the first tube body 102. Upon identifying this empty-tube condition, the detection unit triggers the circuit board 160 to deactivate the heating element 132, thereby preventing unnecessary heating, avoiding potential overheating of an empty tube, and conserving battery power. This coordinated control ensures that heating occurs only when liquid is present and only when required, thereby enhancing safety, energy efficiency, and overall user experience.

Furthermore, in an embodiment, the dispensing head 100 further allows the user to manually adjust the heating temperature of the heating element 132 to achieve a desired level of warmth for the skincare product. The circuit board 160 may be provided with a multi-level temperature control interface, such as a touch button, toggle switch, or digital control module, enabling the user to select from predetermined temperature settings or fine-tune the temperature within a defined safe range. Upon user selection, the circuit board 160 regulates the power supplied to the heating element 132 and cooperates with the temperature sensor to maintain the selected temperature with high accuracy. This manual temperature-adjustment feature provides flexibility for different skin sensitivities, seasonal variations, or product viscosities, ensuring that the skincare formulation is dispensed at a temperature that best suits the user's comfort and therapeutic preference.

The present invention provides a compact, efficient, and user-friendly multifunctional dispensing head that integrates localized heating, precise temperature control, and stimulation features within a single detachable dispensing head. By heating only the liquid present in the flow channel, the device significantly reduces waiting time, preserves the integrity of heat-sensitive cosmetic formulations, and ensures safe, comfortable, and effective application. The multi-layer tube assembly, along with sealing structures and an elastic clamping member, enhances thermal efficiency, prevents leakage, and protects the user from accidental contact with heating elements. Stimulation components, such as massage heads, thermal therapy, or phototherapy units, further enhance treatment outcomes, promoting better absorption and a more enjoyable skincare experience.

This invention has broad applicability in the cosmetic and personal care industry, particularly for premium skincare products requiring precise temperature control during application. It can be used in handheld dispensers for serums, creams, or oils, integrated into luxury beauty devices, or adapted for therapeutic skincare applications in spas, clinics, and at-home treatments, providing manufacturers with a competitive advantage in delivering advanced, user-friendly, and multifunctional cosmetic dispensing solutions.

Various modifications to these embodiments are apparent to those skilled in the art from the description and the accompanying drawings. The principles associated with the various embodiments described herein may be applied to other embodiments. Therefore, the description is not intended to be limited to the embodiments shown along with the accompanying drawings but is to provide the broadest scope consistent with the principles and the novel and inventive features disclosed or suggested herein. Accordingly, the invention is anticipated to hold on to all other such alternatives, modifications, and variations that fall within the scope of the present invention and appended claims.

1. A therapeutic dispensing head for delivering a skincare liquid, comprising:
a first tube body defining a flow channel configured to receive the skincare liquid;
a second tube body disposed around at least a portion of the first tube body, the second tube body and the first tube body together forming a multi-layer tube assembly;
a heating element arranged within or adjacent to the flow channel and configured to heat only the skincare liquid present within the flow channel;
a sealing ring positioned between the first tube body and the second tube body to reduce leakage of the skincare liquid; and
an elastic clamping member positioned at a lower end of the multi-layer tube assembly, the elastic clamping member configured to secure a heating lead and to clamp the first tube body and the second tube body together.

2. The therapeutic dispensing head of claim 1, wherein the heating element comprises a resistive heating wire, a film heater, or a micro-heating chip.

3. The therapeutic dispensing head of claim 1, wherein the heating element is positioned in contact with an outer surface of the first tube body to promote rapid thermal conduction.

4. The therapeutic dispensing head of claim 1, wherein the sealing ring comprises an O-ring, gasket, or elastomeric sealing component.

5. The therapeutic dispensing head of claim 1, wherein the elastic clamping member further comprises a lead-receiving groove configured to hold the heating lead in position.

6. The therapeutic dispensing head of claim 1, further comprising a stimulation unit arranged at an upper end of the multi-layer tube assembly.

7. The therapeutic dispensing head of claim 6, wherein the stimulation unit comprises a metallic, ceramic, or polymer applicator configured to contact a user's skin.

8. The therapeutic dispensing head of claim 1, wherein the second tube body is composed of a thermally insulating material configured to retain heat within the first tube body.

9. A therapeutic dispensing head configured to convey a skincare liquid, comprising:
a first tube body defining a first flow channel for passage of the skincare liquid;
a second tube body arranged around at least a portion of the first tube body and defining at least one secondary flow channel adjacent to the first flow channel;
a sealing structure positioned between the first tube body and the second tube body to reduce leakage between the first flow channel and the second flow channel;
a heating element arranged within or adjacent to the at least one secondary flow channel and configured to heat the skincare liquid present within the first flow channel; and
an elastic clamping member arranged at a lower end of the second tube body and configured to hold the first tube body in position within the second tube body.

10. The therapeutic dispensing head of claim 9, wherein the secondary flow channel is configured to accommodate the heating element, and the skincare liquid passes through a conduction channel of the heating element.

11. The therapeutic dispensing head of claim 9, wherein the sealing structure comprises an annular sealing ring positioned between the first tube body and the second tube body.

12. The therapeutic dispensing head of claim 9, wherein the elastic clamping member comprises a ring-shaped elastic structure configured to exert radial pressure on the first tube body.

13. The therapeutic dispensing head of claim 9, wherein the first tube body and the second tube body together form a multi-layer tube assembly configured to guide the skincare liquid toward an outlet region.

14. A therapeutic dispensing head for delivering a skincare liquid, comprising:
a flow channel configured to receive the skincare liquid;
a tube assembly comprising at least a first tube body forming the flow channel and a second tube body arranged around the first tube body;
at least one sealing component disposed between the first tube body and the second tube body to reduce leakage of the skincare liquid;
a heating element arranged within or adjacent to the flow channel and configured to heat the skincare liquid;
a temperature sensor and a circuit control module configured to regulate operation of the heating element; and
a clamping or retention member configured to secure a heating lead associated with the heating element.

15. The therapeutic dispensing head of claim 14, wherein the temperature sensor comprises a thermistor or a temperature-sensitive integrated circuit.

16. The therapeutic dispensing head of claim 14, wherein the circuit control module is configured to regulate the heating element based on a predetermined temperature range.

17. The therapeutic dispensing head of claim 14, wherein the clamping or retention member comprises an elastic ring, groove, or support bracket configured to hold the heating lead.

18. The therapeutic dispensing head of claim 14, further comprising a stimulation unit arranged at a dispensing end of the flow channel.

19. The therapeutic dispensing head of claim 14, further comprising a battery and a circuit board disposed within a cover, wherein the battery is electrically connected to the circuit board and the circuit board is electrically connected to the heating element.

20. The therapeutic dispensing head of claim 19, wherein a switch button is arranged on an exterior surface of the cover and is configured to control electrical power supplied from the battery to the heating element.
